(19) United States
(12) Patent Application Publication
(10) Pub. No.: US 2010/0159046 A1
(43) Pub. Date: Jun. 24, 2010

(54) MINIMAL TISSUE ATTACHMENT IMPLANTABLE MATERIALS

(75) Inventors: Jeremy Harris, Furlong, PA (US); Constance Ace, Whitehouse, NJ (US); Gonzalo Serafica, Newtown, PA (US)

Correspondence Address: FOLEY AND LARDNER LLP;SUITE 500, 3000 K STREET NW, WASHINGTON, DC, 20007, US

(73) Assignee: Xylos Corporation

(21) Appl. No.: 12/496,143

(22) Filed: Jul. 1, 2009

Related U.S. Application Data
(60) Provisional application No. 61/193,734, filed on Dec. 19, 2008.

(51) Int. Cl. A61K 31/717; A61K 35/66

(52) U.S. Cl. 424/780; 514/57

(57) ABSTRACT

A method for minimizing tissue adhesion at an injured site is provided, the method comprising applying a biocellulose material to the injured site, whereby the adhesion of the tissues at the injured site is minimized, wherein the biocellulose material is at least partially dehydrated. Another embodiment provides an implantable material, which effectively prevents cell adhesion and has desirable mechanical properties.

### RELATED PATENT APPLICATIONS

This application claims priority to U.S. Provisional Application Ser. No. 61/193,734, filed Dec. 19, 2008, which is incorporated herein by reference in its entirety

### BACKGROUND OF THE INVENTION

All the references cited in this Specification are incorporated by reference in their entirety. Unless otherwise specified, “a” or “an” means one or more.

The formation of adhesions following surgery or trauma is undesirable, and numerous materials have been used to prevent the formation of such adhesions, including oxidized cellulose, alginates, chitosan, fibrin, collagen, hyaluronic acid and various synthetic polymers. The main function of these adhesion barrier materials is to prevent both the adhesion of tissue to the material and to the surrounding tissue. Such adhesion can result in adverse consequences, such as scars or permanent damages to the tissue or organs. For example, oxidized cellulose (INTERCEED™, Ethicon, Somerville, N.J.) is a commercial product used in gynecologic surgery to prevent adhesions to the fallopian tubes and ovaries, thereby reducing post-operative pelvic pain and minimizing the risk of infertility due to surrounding tissue adhesions to the fallopian tubes, ovaries and uterus. Another material using hyaluronic acid and carboxymethyl cellulose (Seprafilm™, Genzyme Tissue Repair Inc.) is available to prevent adhesions during general surgery in various areas of the body. A synthetic material made of polylactide (OrthoWrap™, Mast Biosurgery Inc.) has been recommended for use to minimize tissue attachment during orthopedic procedures to prevent adhesion of bone and soft tissue to the implant material. Naturally occurring biopolymers have been described, including chitosan (U.S. Pat. No. 6,150,581), hyaluronic acid (U.S. Pat. No. 6,630,167), alginates (U.S. Pat. No. 6,693,089) and fibrin (U.S. Pat. No. 6,965,014). The use of these naturally occurring biopolymers suggests that highly hydrophilic materials may be used as adhesion barriers or films that minimize tissue attachment.

Cellulose is one of the most abundant biopolymers and is produced by plants and microorganisms. It has been used as starting materials for various implantable medical devices, such as a hemostatic agent (SURGICEL™, Ethicon, Somerville, N.J.), soft tissue reinforcement (Xylos Corporation), and adhesion barriers (INTERCEED™, Ethicon, Somerville, N.J.). Recently, these commercially available oxidized celluloses have been applied to prevent peritendinous fibrotic adhesions (Temiz et al, International Orthopedics 2008 32:389-394) and potentially in pericardial applications (Bicer et al., J of Int'l Med. Res. 2008 36(6) 11). Various research groups have also demonstrated the biocompatibility of unoxidized cellulose when implanted in various areas of the body, including bone and muscle. (Pajulo et al., J. Biomed. Mat. Res. 1996, 32, 439-446). The biological behavior of cellulosic materials during implantation was also described in a rabbit model by Barbie et al., Clinical Materials 1990 251-258). Other investigators have studied tissue biocompatibility of cellulose and its derivatives (Miyamoto et al., J. Biomed. Mat. Res. 1989, 23, 125-133), and have investigated specific applications for the material. Most of the earlier cellulose research has been performed using cotton derived, regenerated or viscose cellulose.

The use of cellulose from other sources, such as microbial cellulose, has also been investigated. For example, the use of microbial cellulose in the medical industry was initially limited to liquid loaded pads (U.S. Pat. No. 4,588,400), skin graft or vulnerary cover (U.S. Pat. No. 5,558,861), wound dressings (U.S. Pat. No. 5,846,213), and other topical applications (U.S. Pat. No. 4,912,049). The implantability of the microbially-derived material was first studied for use as a dura substitute (Mello et al., Journal of Neurosurgery 1997, 86, 143-150), which was later expanded in U.S. Pat. No. 7,374,775. Recently, in vivo implantation of bacterial cellulose was disclosed by Helenius et al. (Journal of Biomedical Materials Research 2006, 76A; 431-438), wherein the material was shown to have good biocompatibility. The same group also suggested the use of microbial as a potential scaffold for tissue engineering of cartilage (Svensson et al., Biomaterials 2005, 26, 419-431). Microbial cellulose and other biomaterials have been examined as implant materials or as buttresses for suture augmentation for tendon repair and reattachment (Kummer et al., J. Biomed Mater Res Part B: Appl Biomater 2005, 74B: 789-791). U.S. Pat. No. 6,599,518 discloses the use of solvent dehydrated microbially derived cellulose for in vivo implantation and as medical devices. U.S. Patent Application No. 2003/00131163 describes a method for producing shaped microbial cellulose and a device thereof for use as blood vessels and microsurgery applications. Also, a composite biocompatible hydrogel, which can support cell colonization in vitro, has a Young's modulus of at least 10 GPa, and comprises a porous bacterial cellulose and a calcium salt, for use as a bone or cartilage implant is described in U.S. Patent Application No. 2004/0096509. Combining microbial cellulose with synthetic polymers to form composites has also been reported by Wan in U.S. Patent Application No. 2005/0037082. Finally, chemical modification of microbial cellulose to render it bioresorbable has been reported recently in U.S. Patent Application No. 2007/0213522.

Microbial cellulose possesses natural properties, such as high hydrophilicity and microfibril assembly, rendering it potentially suitable for applications such as an adhesion barrier. However, the resultant implantable material should be tailored such that the material is effective in providing minimum tissue attachment (MTA). Therefore, a need exists to produce a microbial cellulose material with a desirable level of hydrophilicity, a capability to carry a bioactive agent, such as drugs, and which can prevent cell and/or tissue adhesion and provide minimal tissue attachment.

### BRIEF DESCRIPTIONS OF THE FIGURES

FIG. 1 shows a scanning electron micrograph (SEM) of the surface of a microbial cellulose sample (“Sample 530”), which has high tensile strength and is capable of minimizing tissue attachment.

FIG. 2 shows a SEM of the surface of a conformable microbial cellulose sample (“Sample 50”) capable of minimizing tissue attachment.

### SUMMARY OF THE INVENTION

One embodiment provides a microbial cellulose material, wherein the implantable device can minimize the attachment of tissues to each other. An alternative embodiment provides an implantable microbial cellulose material, wherein the material is produced with the desirable mechanical (e.g. tensile, suture pull-out strength, and/or stiffness) while maintaining its structural (e.g., planar isotropic non-woven mesh with lamellar superstructure) properties to provide a cell-impermeable surface and prevent the formation of cell and/or tissue adhesions when implanted in vivo. Additional embodiments provide methods for producing these materials and for using these materials.

Another embodiment provides microbial cellulose materials, particularly Acetobacter xylinum cellulose, used as an implantable medical device for minimizing unwanted cell and/or tissue attachment and/or adhesions that occur as a result of trauma or surgical insult. Another embodiment is used as an adhesion barrier and/or as a drug delivery carrier for the prevention of post-surgical cellular adhesions. The implantable materials described herein can be optimized for a wide range of surgical procedures, including management of adhesions in abdominal, cardiothorasic, orthopedic, and/or neurosurgery procedures, and/or for the protection of soft tissues and the establishment of a surgical plane of dissection in, for example, a spine at the site of implantation and/or the surgical site. Embodiments can also include a family of devices having a wide range of properties suitable for high strength and/or high conformability applications.

In one embodiment, a method for minimizing tissue adhesion at an injury site is provided, the method comprising applying a biocellulose material to the injury site, whereby the adhesion of the tissues at the injury site is minimized, and wherein the microbial cellulose material is at least partially dehydrated.

In another embodiment, a method for producing microbial cellulose to be used as an adhesion barrier and to minimize cell and/or tissue attachment is provided. The method comprises: (i) providing a biocellulose material; (ii) oxidizing the biocellulose material; (iii) de-pyrogenating the biocellulose; and (iv) dehydrating the biocellulose material. The material produced can be partially dehydrated to control the physical properties. Exposure to various dehydration conditions, such as temperatures below the freezing temperature of the fluid in the sample or under ambient conditions, can be employed. The effect of the drying process on tensile strength, stiffness, and suture pull-out strength characteristics is also shown. Desirable properties, such as conformability, high pliability, and the ability to deliver bioactive agents, such as drugs, are also described. Moreover, the non-limiting examples herein demonstrate minimizing tissue attachment and preventing post surgical adhesions in vivo using microbially-derived cellulose.

An implantable biocellulose material is provided in one embodiment, which minimizes tissue attachment at an injury site in a subject, wherein the biocellulose material is at least partially dehydrated and wherein the biocellulose material is implanted at the injury site.

### DETAILED DESCRIPTION

### Biocellulose Materials

In one embodiment, the method for producing materials that can minimize tissue attachments using microbially-derived cellulose is provided. However, the cellulose material can be selected from any cellulose form, including powders, sponges, knitted, woven and non-woven fabrics made of cotton, rayon, or combinations thereof. Also included in the types of cellulose are cellulose films, cellulose paper, cotton or rayon balls, fibers of cotton or regenerated cellulose, or a pellicle of cellulose produced by a microorganism, such as a bacterium, such as Acetobacter xylinum.

These materials can act as adhesion barriers between adjoining tissues by minimizing cell infiltration and/or permeation. Such ability to minimize cell and/or tissue attachment may be attributed to different properties of the material, such as their small effective pore size. The materials can be produced and subsequently processed by controlled dehydration in order to preserve the desirable pore size while preserving its pliability and lubricity. Dehydration can be accomplished by either using a series of solvent exchanges followed by a controlled drying process, mechanical pressing, drying with supercritical carbon dioxide, or thermally modifying the sample (see e.g., U.S. patent application Ser. No. 10/920,297) to preserve the hydrophilicity of the materials to maintain pliability while producing sheets with adequate strength for a particular application.

Various methods for producing the raw microbial cellulose material can involve a static or rotating disk method, and/or agitated cultures. The resulting material from fermentation can be subsequently “cleaned” and washed using a solution, such as a caustic solution, such as a concentrated sodium hydroxide solution. Although minute amount of organic residues (e.g., cells or cell fragments) can be present, the cleaning process removes substantially all the microbial cells and excess medium and renders the material non-pyrogenic. The cleaning process can further include a step of whitening the biocellulose material with an agent. The whitening agent used to further assure the cleanliness of the biocellulose can be an oxidizing agent such as hydrogen peroxide.

One advantage of oxidizing the material is to improve the bioresorbability of the material via an oxidizing agent. The oxidizing agent can be, for example, sodium periodate, nitrogen tetroxide, or a combination thereof. In one embodiment, wherein nitrogen tetroxide is used, prior to oxidation, the material is preferably in a dry state to prevent water from quenching the nitrogen-tetroxide. The material can be brought into the dry state by, for example, another suitable drying step, which can be any of the drying steps mentioned below. The ratio of the oxidizing agent in the solution to cellulose can vary, depending on, for example, the desired oxidation level. For example, the ratio can be less than about 20, such as less than about 10, such as less than about 8, such as less than about 5, such as less than about 1, such as less than about 0.5, such as less than about 0.01, such as less than about 0.08, such as less than about 0.05.

### Dehydration

Microbial cellulose can have high water content, in excess of 60%, such as in excess of 80%, such as in excess of 90%, such as in excess of 95%. In order to achieve the desired material properties for the anti-adhesive barrier material, some or all of the water present in the material can be removed. For example, in one embodiment, the cleaned microbial cellulose material is further dehydrated, or “dried.” The dehydration of the biocellulose material can be accomplished using different methods. For instance, the dehydration method can involve solvent dehydration, supercritical drying (e.g., supercritical drying with carbon dioxide), lyophilization, controlled drying, mechanical pressing, thermal dehydration (or “thermal modification”), or combinations thereof. The material produced can be partially dehydrated or substantially fully dehydrated to control the physical properties. For example, more than 50% of the water can be removed, such as more than 60%, such as more than 80%, such as more than 90%, such as more than 95%, such as more than 99%, such as more than 99.5% of the water.

In one embodiment, the use of solvents to exchange, and thus to remove, the water in the raw cellulose can be employed before the drying step. Various solvents, including methanol, ethanol, isopropyl alcohol, or combinations thereof, can be used for the water-solvent exchange. It is important that in the dehydration steps at least some of the absorption capability of the material is preserved so that the material can remain pliable and at the same time maintain its small pore size to prevent cell infiltration. In one embodiment, this can be achieved by controlled dehydration at substantially constant humidity.

In one embodiment, the controlled drying process (CD) can be performed as follows. Prior to drying, the liquid composition of the cellulose sample may comprise water, methanol, ethanol, isopropanol, or a combination thereof. The wet pellicle is placed in a closed chamber which allows for controlled gas flow in the chamber. An inert gas, such as nitrogen, can be flowed through the chamber to control the relative vapor concentration therein. The flow rate of the inert gas can control the concentration of the solvent in the vapor, thereby controlling the rate at which the cellulose dries. By controlling the drying rate, the liquid content of the microbial cellulose can be adjusted gradually over time in the drying chamber. In one embodiment, the mass of the microbial cellulose can remain substantially constant during the drying process, producing materials with cellulose concentrations in the material anywhere from between about 2% and about 6%, such as 5% at the start of the process up to between about 98.5% to about 99.5%, such as 99% at the end of the drying process. The material can have any suitable configuration, such as a sheet, pad, pellicle, or tube.

By adjusting the drying times, the microbial cellulose material can have different microstructures, resulting in different swelling behavior during rehydration. For example, some dehydrated materials exhibit complete rehydration within minutes, while some others take over an hour, such as over a day or even a week to be completely rehydrated. Alternatively, the dehydrated materials can reabsorb fluid gradually, minimizing swelling after an extended period of soaking time. As a result, not to be bound by any particular theory, the rate of swelling and increase in the thickness of the microbial cellulose material (e.g., sheet) can indicate how open the structure is after dehydration. In one embodiment, the desired materials can maintain their thickness while absorbing a small amount of fluid to make them pliable after being soaked for 15-30 minutes.

The material prior to or after dehydration can be further subjected to chemical modification depending on the product requirements. Such chemical modifications may include cross-linking of the cellulose fibers to enhance the physical properties, such as the mechanical strength, and/or oxidation of the cellulose to make the material bioresorbable. The oxidation levels can be varied depending on the desired resorption rate, or time for the material to be resorbed by the body. The degradation (or resorption) times of the resulting materials can range from weeks, such as more than about 1 week, such as more than about 4 weeks, to more than about one year, such as to more than about 2 years.

### Microstructures

The biocellulose material described herein is generally porous. The pores of the biocellulose material can have any size and form. For example, the pores of the biocellulose can be cylindrical, spherical, elliptical, or combinations thereof. In one embodiment, the pore size of the biocellulose material is such that the cells cannot easily infiltrate the material, thus rendering the biocellulose material substantially cell impermeable. The materials need not be entirely impermeable to the cells. For example, the material can be substantially cell-permeable or semi cell-permeable. For example, a very small number of cells might be present in the biocellulose.

Various methods can be used to characterize the pore size of the biocellulose material described herein. For example, the pore size can be described by the “effective pore size,” which can be defined as the maximum size of an object that is allowed to pass through the material. For example, if the object is a biological cell, which generally has a size of about 1-10 microns (or greater), and no other entities larger than the biological cell can pass through the cellulose material, the effective size of the cellulose material is deemed to be the size of the biological cell.

The effective pore size of the biocellulose material product can vary. In one embodiment, the effective pore size can be less than or equal to about 10 microns, such as less than or equal to about 5 microns, such as less than or equal to about 2 microns, such as less than or equal to about 1 micron, such as less than or equal to about 0.5 microns, such as less than about 0.3 microns, such as less than about 0.1 microns.

One feature of the biocellulose material described herein, particularly the methods of making thereof, is that the microstructure of the resulting biocellulose material is comparable to that of the raw biocellulose material prior to the processing steps described above. For example, if the natural, unprocessed biocellulose pellicle has an effective pore size of less than about 5 microns, such as less than about 1 micron, the effective size of the biocellulose material after the processing steps described above can also have a effective pore size of about less than about 5 microns, such as less than about 1 micron. Alternatively, if the natural cellulose pellicles have a nonwoven like microstructure, the resulting, processed biocellulose material can also have a nonwoven like microstructure.

### Biological Agent

The dehydrated biocellulose can also be impregnated or coated with various biological agents, such as biologically active agents, to enhance its biological properties. A host of biologically active agents, such as drugs, peptides, antimicrobials, proteins, including fibrin, and a variety of growth factors, can be impregnated into or coated onto the resulting biocellulose material prior to implantation of the cellulose material. The biologically active agent can be autologous, allogenic, xenogenic, synthetic, or combinations thereof. The release of such bioactive agents into the subject, in whom the biocellulose material is implanted, can also be controlled by altering the microstructure depending on the desirable delivery rate for a specific application. A sample list of active agents that can be incorporated into the microbial cellulose include Bone Morphogenetic Proteins (BMP), platelet derived growth factors (PDGF), transforming growth factors (TGF), growth and differentiation factors (GDF), insulin-like growth factor (IGF), epidermal growth factor (EGF), demineralized bone matrix (DBM), Factor VIII, or combinations thereof.

Depending on the intended application, also suitable can be the use of viable differentiated and undifferentiated cells for the growth of biological soft tissues, such as connective tissues, including bone, spine, cartilage, ligaments, tendons, skin, vessels, such as blood vessels, fallopian tubes, or organs, such as heart, ovary, uterus, or combinations thereof. These agents and/or cells can be added to or coated on the surface of the microbial cellulose material. The biocellulose material can also be free of the biologically active agent. For example, in one embodiment, the biocellulose is substantially free of biologically active agent, such as an peptide, such as an adhesion peptide, or signaling molecules such as growth factors.

### Mechanical Properties

The resulting material can be further tested for its physical, chemical, and/or biological properties to determine its use as an adhesion barrier and to demonstrate its ability to minimize cell and/or tissue attachment. One feature of the biocellulose material and the processes of making the material is that, while some characteristics such as the microstructure (e.g., effective pore size) of the natural biocellulose material can be substantially preserved through the processing steps, the mechanical properties of the biocellulose material product can be controlled and/or improved over the biocellulose material in its natural state.

An in vitro testing can include physical testing of the product for its mechanical properties, such as tensile strength and/or suture pull-out resistance. One desirable property with respect to the tensile strength is for the material to hold its integrity both prior to implantation and while in service post implantation in the body in vivo. In one embodiment, the resulting biocellulose material has a tensile strength of from about 0.5 N to about 400 N, such as 1 N to about 300 N. Generally, a value of greater than about 2 N, such as about 3 N, such as about 6 N, such as about 9 N, such about 12 N, such as about 20 N, for the tensile strength is desirable for non-load-bearing applications, and over about 100 N, such as over about 150 N, such as over about 200 N, such as over about 250 N, such as over about 275 N, such as over about 300 N, for the tensile strength is desirable for load-bearing applications. Non-load bearing applications include, for example, a tissue wrap membrane for reducing attachment of tissues, such as pericardial tissues, following surgery, such as a cardiothoracic surgery. Load-bearing applications include, for example, a reinforcement matrix used to enhance suture security in the surgical repair of a soft tissue, such as tendons, in rotator cuff reconstruction and/or repair.

The biocellulose material can also have a wide range of suture pull-out strength, depending on the desired application of the material. For example, the suture pull-out strength can be between about 0.1 N to about 20 N, such as 0.3 N to about 15 N. In one embodiment, a biocellulose material that acts to reinforce tissue can have a suture pull-out strength of greater than about 6 N, such as greater than about 9 N, such as greater than about 12 N, such as greater than about 15 N, whereas in non-load-bearing applications the material can have a suture pull-out strength of less than about 3 N, such as less than about 2 N, such as less than about 1 N, such as less than about 0.5 N, such as less than about 0.3 N, to maintain a tack suture.

The biocellulose material can have a wide range of stiffness, depending on the desired application of the material. For example, the suture pull-out strength can be between about 0.5 N to about 100 N, such as 1 N to about 40 N. In one embodiment, the cellulose material that is used for a non-load-bearing application can have a stiffness that is less than about 10 N, such as less than about 5 N, such as less than about 3 N, such as less than about 2 N, such as less than about 1 N. In one alternative embodiment, wherein the biocellulose material is used in a load-bearing application, stiffness of the material is greater than about 10 N, such as greater than about 20 N, such as greater than 40 N, such as greater than about 60 N.

The resulting biocellulose material need not only be used in a load-bearing or non-load-bearing application. For example, because of the biocellulose material described herein has a minimum tissue attachment ability, the material can be used as a marker at a surgical site, since the tissues around the surgical site cannot be allowed to adhere, thereby to masking the location of the surgical site. For example, in one embodiment, the biocellulose is used to establish a post-operative plane of dissection at, for example, a spine. The biocellulose is intended to provide enhanced access to the tissue, or a specific portion of the tissue, at the surgical site, since the site can be substantially free of covering adhered tissue. Such access can facilitate (and thus improve the ease of) blunt dissection in subsequent surgical approaches to the same anatomical location.

### Bioresorbability

The biocellulose material described herein is generally biocompatible. It can also be bioresorbable. The material's degradation in various solutions can be measured to estimate how long the material can remain intact in the body. The bioresorption time (or “bioresorption rate”) of the biocellulose material described herein can be, for example, between about less than about 7 days to more than about 3 years, such as between about 30 days and about 2 years. The material in non-load-bearing applications, wherein the material can be used mainly to minimize the formation of post-operative adhesions, may have short resorption times, such as less than about 30 days, such as less than about 14 days, such as less than about 7 days. Alternatively, in other applications, wherein the strength is desired to be maintained, the material may have a resorption time of greater than 0.5 years, such as greater than 1 year, such as greater than 2 years.

### Animal Studies

Various animal models, including a uterine horn model in rabbits in a uterine horn model (Wiseman et al., J. Inv. Surg. 1999, 12:141-146) and a cecal abrasion model in rats in a cecal abrasion model (Avatal et al., Dis Colon Rectum 2005, 48, 153), can be employed to illustrate the material's ability to minimize tissue adhesions in vivo. The cecal abrasion model in the rats, for example, can illustrate the materials' ability to prevent adhesion formation between the cecum and abdominal wall. In this model, abrasions are created between the cecum and the corresponding area of the abdominal wall. The device is placed to prevent the abraded regions from overlapping. After a desired time interval, the surgical site is evaluated and adhesion formation is graded based on adhesion tenacity and the percent area of the device covered by adhesions. The method of evaluating the tenacity is generally known in the art. An illustrative non-limiting example of such an evaluation is provided in Example 10 in a later section. In one embodiment, the tenacity for tissue and/or cell adhesion of an implantable material comprising the cellulose material described above is less than about 2.5, such as less than about 2, such as less than about 1.5, such as less than about 1.0, such as less than about 0.5, such as about 0.

### Applications

The resultant biocellulose samples can be in the form of cellulose sheets, such as medical grade cellulose sheets. The biocellulose material, after the fabrication and cleaning and/or oxidation process, can be punched, packaged and gamma sterilized. The biocellulose material can be a part of an implant for repairing or augmenting tissues, such as connective tissues, such as including bone, cartilage, ligaments, tendons, skin, vessels, such as blood vessels, spines, or organs, or combinations thereof. The implant can be used in a subject in need thereof, and the subject can be an animal, such as a mammal, including a human. The biocellulose material can be a material that is (bio)resorbed by the body in a short time and in the meantime promotes tissue healing by formation of new tissues instead of tissue adhesion. Alternatively, the biocellulose material can serve as a tissue anchor, which is bioresorbed at a slower pace than that in the previous embodiment. In one embodiment, a microbial cellulose material is applied to an injury site. The injury can be a result of surgical or traumatic insult, lesion, abrasion, and the like caused by either purposely created injury or accidentally (and naturally) occurring injuries. The material can also be used in cardiovascular repairs, such as heart valve repairs (to prevent, for example, pericardial adhesions), fallopian tubes, ovaries, and/or uterus repairs.

### NON-LIMITING WORKING EXAMPLES

The following examples are given to illustrate the present invention. It should be understood, however, that the invention is not to be limited to the specific conditions or details described in these examples. Throughout the specification, any and all references are specifically incorporated into this patent application by reference.

### Example 1

### Production of Microbial Cellulose by Acetobacter Xylinum

This example describes the production of microbial cellulose by Acetobacter xylinum suitable for use in preparing a minimum tissue attachment (MTA) material. The production involved the inoculation of sterilized medium with A. xylinum from a propagation vessel prior to incubation. The inoculated medium was then used to fill bioreactor trays to a fixed volume, including 30, 50, 110, and 530 g (and thus “Sample 30,” Sample 50,” Sample 110,” and Sample 530”). The fill volume refers to the amount of inoculated media added to a bioreactor tray with a maximum volume of 590 g. A higher fill volume represents a finished product with a higher cellulose content. The trays were covered with a plastic sheet with aeration ports added for oxygen exposure during growth. Trays were then incubated under static conditions at a fixed temperature of 30° C. until optimal growth was achieved (4 to 35 days, depending on the initial volume of medium.)

The microbial cellulose pellicles were harvested and subjected to a weight check to verify that growth was achieved according to an established weight and/or cellulose specification.

### Example 2

### Processing of Microbial Cellulose

The microbial cellulose produced according to Example 1 was subjected to a series of chemical processes to clean and whiten its appearance. Prior to chemical processing, the pellicles were pressed with a pneumatic press to achieve the desired extraction weight.

The pressed cellulose pellicles underwent chemical processing that included a dynamic soak in a heated tank of caustic solution for approximately one hour to depyrogenate the material. This chemical process was followed by a continuous rinse with filtered water to remove the caustic solution from the processed pellicles. Subsequent to rinsing, an additional chemical oxidizing agent, hydrogen peroxide was used to whiten the pellicles. Following chemical processing, the microbial cellulose films were again subjected to dehydration in a pneumatic press to achieve a pre-designated weight or thickness and then subjected to post-chemical processing steps, as described below.

### Example 3

### High Strength, Non-Resorbable Device

The process for fabricating a biocellulose material for non-resorbable implant, which generally needs high strength, began with fabricating a Sample 530 material as described in Examples 1 and 2, followed by a solvent dehydration process to remove a portion of the water present in the pellicles. Following solvent dehydration the pellicles were mechanically dehydrated to a pre-designated weight and controlled dried to a level of <10% fluid content. A final step of a rehydration in 0.125% H₂O₂ was performed prior to punching, packaging, and sterilization.

### Example 4

### Highly Conformable, Non-Resorbable Devices

The process for fabricating a biocellulose material for non-resorbable implant, which generally needs a high level of conformability, began with producing a Sample 30 material as described in Examples 1 and 2, followed by a thermal modification dehydration process to remove a portion of the water present in the pellicles. Following thermal modification the pellicles were warmed to greater than about 20° C. The biocellulose material samples were then punched, packaged, and gamma sterilized.

### Example 5

### High Strength, Resorbable Devices

The process for resorbable devices involved an additional step of oxidation to render a high strength microbial cellulose bioresorbable. The Sample 530 materials, as described in Examples 1 and 2, in the form of pellicles were immersed in a sodium periodate solution, resulting in a periodate to cellulose ratio of about 0.08. The pellicle samples were oxidized for about 16-18 hours at about 23±2° C., followed by a water rinse process to remove unreacted periodate. The oxidized, and thus resorbable, samples of those described in Example 1 are hereafter labeled as “Sample 30-R,” Sample 50-R,” Sample 110-R,” and Sample 530-R.” Once the excess periodate was removed, the oxidized pellicles were mechanically dehydrated to remove >50% of the residual water from the pellicles. The pellicles then underwent multiple solvent exchanges with methanol to remove >95% of the residual water. The pellicles were then mechanically dehydrated before a CD process as described in Example 3. The pellicles were briefly rehydrated in methanol before the final drying step with supercritical carbon dioxide. The pellicles were wrapped in a polypropylene mesh and placed in a supercritical fluid exchange system (150 SFE System, Super Critical Fluid Technologies, Inc., Newark, Del.). The vessel was brought to 4000 psi and 40° C. and a series of static/dynamic cycles were performed until complete methanol removal was achieved. Following the supercritical process, the oxidized material was removed from the vessel in a dry form.

### Example 6

### Highly Conformable, Resorbable Devices

The process for resorbable devices involved an additional step of oxidation to render a conformable microbial cellulose bioresorbable. The microbial cellulose (Sample 50), as described in Examples 1 and 2, was immersed in a sodium periodate solution resulting in a periodate to cellulose ratio of about 8. The pellicle samples were oxidized for about 16-18 hours at about 23±2° C. before a water rinse process to remove the unreacted periodate. The oxidized sample is thus “Sample 50-R.” Following the rinse process the pellicles underwent multiple solvent exchanges with methanol to remove more than about 95% of the residual water from the pellicles. Pellicles were then mechanically dehydrated before a drying step with supercritical carbon dioxide, as described in Example 5.

### Example 7

### Nitrogen Tetroxide Oxidation of Microbial Cellulose

An alternative oxidation method using nitrogen tetroxide was used to render microbial cellulose bioresorbable. Prior to oxidation, the material was in a relatively dry state to prevent the water from quenching the nitrogen tetroxide. This was an additional step prior to oxidation that was not performed when using the previous periodate oxidation method. Once the material was dried, the material was soaked in a perfluorinated tertiary amine solvent, and a pre-determined amount of nitrogen tetroxide was added to the reaction vessel. The reaction time was set for up to about 24 hours, after which the cellulose sheets were washed with methanol to remove any excess oxidizing agent and solvent. The material was further soaked in methanol prior to final dehydration with supercritical drying as described below.

### Example 8

### Mechanical Testing of MTA Materials

Tensile, suture pull-out strength, and stiffness were measured to characterize the strength of the various materials produced as described in Examples 3-6. Tensile and suture pull-out strength testing was conducted on 1 cm×4 cm test strips, obtained from Examples 3-6, that were mounted into pneumatic clamps of a United Tensile Tester (Model SSTM-2kN) fitted with either a 100 N or 500 lb. load cell, depending on the samples tested. The gage length for tensile testing of the specimens was 25 mm. The specimens were subjected to displacement at a rate of 300 mm/minute until the specimen failed completely. Failure force was determined from the force-displacement curve at the maximum force (N).

Suture pull-out testing used either 2-0 or 5-0 polypropylene suture (Prolene, Ethicon Inc.) with a curved needle. A single stitch was placed approximately 5 mm from each side and 4 mm from the bottom edge of the specimen. The suture thread was cut to approximately 10 cm. The top end of the specimen was mounted into pneumatic clamps on the United Tensile Tester. Both ends of the suture were mounted into the lower set of pneumatic clamps. The specimens were subjected to a displacement of 300 mm/minute until the specimen failed completely.

Stiffness testing followed ASTM D4032-94, Standard Test Method for Stiffness of Fabric by the Circular Bend Procedure. The probe diameter was 0.500″ and the platform orifice was 0.875″. Cross-head speed was 300 mm/min with a 100 N load cell. A single 4 cm×5 cm device was used for each test measurement.

TABLE 1
Mechanical testing data for non-oxidized and oxidized materials
as described in Examples 3-6.

| | Suture | | |
|---|---|---|---|
| | Pull-Out | Tensile Strength | |
| Sample | Strength (N) | (N) | Stiffness (N) |
| High conformability, | 0.62 ± 0.14 | 9.8 ± 2.2 | 3.1 ± 1.6 |
| non-resorbable | | | |
| High conformability, | 0.40 ± 0.04 | 3.1 ± 1.0 | 1.6 ± 0.7 |
| resorbable | | | |
| High strength, non- | 13.9 ± 3.1 | 203.6 ± 45.6 | 22.0 ± 4.0 |
| resorbable | | | |
| High strength, resorbable | 12.0 ± 3.6 | 276.8 ± 54.2 | 43.0 ± 6.0 |

### Example 9

### Scanning Electron Microscope (SEM) Analysis of MTA Structure

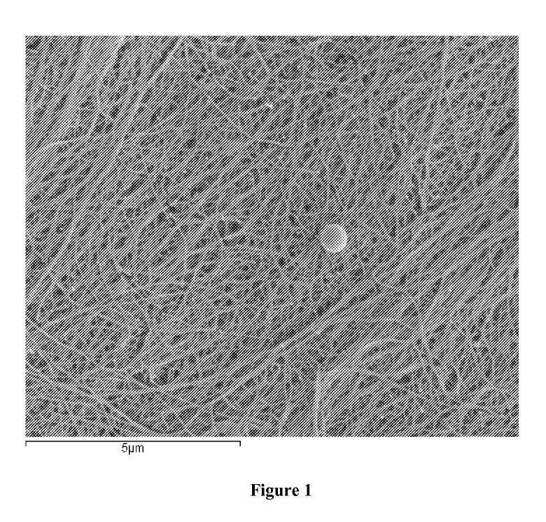

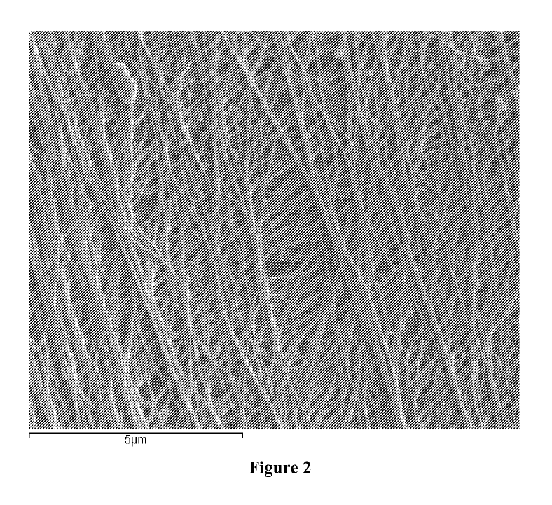

SEM images were taken on the MTA materials produced in accordance with the steps described above to evaluate the surface microstructure and to determine the effective pore size of the material. FIG. 1 shows a SEM image of the surface of “Sample 530,” which has high tensile strength is capable of minimizing tissue attachment. FIG. 2 shows a SEM image of the surface of “Sample 50,” which is highly conformable and is capable of minimizing tissue attachment. Both FIGS. 1 and 2 show that the samples demonstrate a highly nonwoven structure of the biocellulose. The pore size in FIGS. 1 and 2 is about <0.5 μm, which is significantly smaller than most types of cells. The nanoporous structure of the biocellulose limits the ability of cellular in-growth therein, thereby providing a material which exhibits minimal tissue attachments in vivo.

### Example 10

### In Vivo Demonstrations of Minimizing Tissue Attachment

Of the several models available to show the ability of a material to minimize cell adhesions and tissue attachments, the in vivo cecal abrasion model in both rabbit and rats was used to demonstrate the capability of the material described herein in minimizing tissue attachment. The cecal model is a well accepted model for adhesion formation wherein a certain area of the cecum and adjacent abdominal wall of the animal is abraded to promote adhesion formation between the two surfaces. An implant material was introduced in between the abraded tissue surfaces as the test material and each test material was evaluated via two metrics. Approximately 7-15 days after the surgical procedure, the area of the injury was examined and graded with respect to the extent of adhesions formed (tenacity) with a scale of 0 to 3 or 0 to 4, depending on the scoring system used, wherein a score of 0 indicates no interaction between the tissue surfaces and 3 to 4 indicates the strongest adhesions. The area of the adhesions formed, as a percentage of the total area of the injury site, was also evaluated.

### Study One—Rabbit

The first series of studies involved the use of female New Zealand rabbits in groups of four animals, where each animal received one of the four implant materials, Sample 30 (“30”), Sample 30-Resorbable (“30-R”), Sample 530 (“530”), and Sample 530-Resorbable (“530-R”), which were made in accordance with the description above. Each of the implant materials was introduced in between the cecum and the abdominal wall. Three negative control animals, to which no implant was introduced between the injured cecum and abdominal wall, were also used. After 15 days of implantation, the area was examined, and the strength and area of adhesions formed were rated. In the control group, adhesions formed in 100% of the area, and an average tenacity of 3.4 (maximum possible score in this study was 4.0) was observed. The test group which contained a microbial cellulose material (Sample 530) between the cecum and abdominal wall displayed reduced scores of 25% of the total injured area on the abdominal side and about 73% of the area of the cecal side. The tenacity of the adhesions were rated at 2 and 2.3 for the abdominal side and cecal side, respectively. The results of these studies are summarized in Table 2, below. Overall, the test materials displayed a significant reduction in the area involved in adhesion formation and in the strength of the adhesions between the adjoining tissues.

TABLE 2
Results of a study of the effects of various biocellulose materials
in a rabbit cecal abrasion model.

| | | | Abdominal side | |
|---|---|---|---|---|
| | Cecal side | | | Area |
| Sample | Tenacity | Area of Adhesion | Tenacity | of Adhesion |
| Control | 4.0 | 100% | 3.4 | 100% |
| 30 | 1.1 | 28% | 1.5 | 100% |
| 30-Resorb. | 0.5 | 20% | 0.8 | 100% |
| 530 | 2.3 | 75% | 2.0 | 25% |
| 530-Resorb. | 1.2 | 63% | 1.2 | 73% |

Study Two—Rat

The second in vivo demonstration involved the same model except conducted in rats. There were four test groups of various prototypes of the microbial cellulose materials and a control group receiving no implant after the cecal injury. The results were similar to the rabbit study, where the majority of the control group (12 out of 13) displayed strong adhesions involving 75-100% of the area of the injury. In all of the test group animals, where a microbial cellulose device was placed in between the tissue, there was a reduction of adhesions formed and the strength of the adhesions. In one group using Sample 30 oxidized to 80%, only 2 out of 13 showed any visible adhesion and the tenacity of the formed adhesion was very low and involved less than 25% of the area. The non-oxidized version, Sample 110 specimen, as described in Example 5 also minimized tissue attachment in 10 of the 12 samples examined.

These results show that all animals implanted with the materials described above showed a significant decrease in adhesion tenacity, which is the better indicator of the two metrics to evaluate the clinical impact of the cellulose. The use of the material to prevent adhesion formation and minimizing tissue attachments was clearly demonstrated by these example studies. All materials were therefore determined to be effective at decreasing adhesions.

The following embodiments are from the U.S. Provisional Application No. 61/193,734, filed Dec. 19, 2008:

1. A method for minimizing tissue adhesion comprising applying microbial cellulose to a surgical site in a subject in need thereof in order to minimize adhesion between adjacent tissues, wherein the microbial cellulose is dehydrated and has average opening sizes of no more than 10 microns as determined by scanning electron microscopy.

2. A material having high tensile strength of about 100 N and capable of minimizing tissue attachment.

3. A material that is highly conformable with a stiffness of about 2 N and capable of minimizing tissue attachment.

4. A material used for preventing adhesion during tendon repair.

5. A material capable of creating a plane of dissection for spine and other applications.

6. A material that can prevent peritendinous adhesions and pericardial adhesions.

7. The method of embodiment 1, wherein the material is oxidized using nitrogen tetroxide.

8. The method in embodiment 1, wherein the material is oxidized using sodium periodate.

9. The method of embodiment 1, wherein the dehydration of the microbial cellulose is accomplished by a method selected from the group consisting of a solvent dehydration, supercritical drying method, lyophilization, controlled drying under constant humidity and thermal dehydration.

The foregoing description of the embodiments of the invention has been presented for purposes of illustration and description. It is not intended to be exhaustive or to limit the invention to the precise form disclosed, and modifications and variations are possible in light of the above teaching or may be acquired from practice of the invention. The embodiments were chosen and described in order to explain the principles of the invention and as a practical application to enable one skilled in the art to utilize the invention in various embodiments and with various modification are suited to the particular use contemplated. It is intended that the scope of the invention be defined by the claims appended hereto and their equivalents.

What is claimed:

1. A method for minimizing tissue adhesion at an injury site, comprising applying a biocellulose material to the injury site, wherein the biocellulose material is at least partially dehydrated, and whereby the adhesion of the tissues at the injury site is minimized.

2. The method of claim 1, wherein the biocellulose material is produced by a microorganism.

3. The method of claim 1, wherein the biocellulose material has an effective pore size of less than or equal to about 1 micron.

4. The method of claim 1, wherein the biocellulose has at least one of:
(i) a tensile strength of between about 1 N and about 300 N
(ii) a stiffness of between about 3 N and about 40 N
(iii) a suture pull-out strength of between about 0.3 N and about 15 N.

5. The method of claim 1, wherein the biocellulose has a tissue adhesion tenacity of less than about 2.

6. The method of claim 1, wherein the biocellulose is bioresorbable.

7. The method of claim 5, wherein the biocellulose has a bioresorption rate of between about 14 days and about 3 years.

8. The method of claim 1, wherein the at least some of the tissues at the injury site do not attach to the biocellulose material.

9. The method of claim 1, wherein the biocellulose material comprises a biologically active agent.

10. The method of claim 1, wherein the biocellulose material is substantially free of a biologically active agent.

11. A method of making an implantable biocellulose material which minimizes tissue attachment, comprising:
(i) providing a biocellulose material;
(ii) oxidizing the biocellulose material;
(iii) de-pyrogenating the biocellulose; and
(iv) dehydrating the biocellulose material.

12. The method in claim 11, wherein the biocellulose material is oxidized using at least one of (i) nitrogen tetroxide and (ii) sodium periodate.

13. The method of claim 11, wherein the biocellulose has a tissue adhesion tenacity of less than about 2.

14. The method of claim 11, wherein a microstructure of the biocellulose material before step (i) is substantially the same as the microstructure of the biocellulose material after step (iv).

15. The method of claim 11, wherein the step of dehydrating the microbial cellulose is accomplished by a method selected from the group consisting of a solvent dehydration, supercritical drying method, lyophilization, controlled drying under constant humidity and thermal modification.

16. The method of claim 11, further comprising rehydrating the biocellulose material after step (iv).

17. The method of claim 11, wherein the biocellulose material is substantially free of a biologically active agent.

18. The method of claim 11, wherein the biocellulose material has an effective pore size of less than or equal to about 1 micron.

19. The method of claim 11, wherein the biocellulose material has at least one of
(i) a tensile strength of between about 1 N and about 300 N
(ii) a stiffness of between about 3 N and about 40 N
(iii) a suture pull-out strength of between about 0.3 N and about 15 N.

20. An implantable material made according to the method of claim 11.

21. The implantable material of claim 20, wherein material prevents adhesions of at least one of the adjoining tissue or organ at an implantation site.

22. The implantable material of claim 20, wherein the material creates a plane of dissection at an implantation site.

23. An implantable biocellulose material which minimizes tissue attachment at an injury site in a subject, wherein the biocellulose material is at least partially dehydrated and wherein the biocellulose material is implanted at the injury site.

24. The implantable biocellulose material of claim 23, wherein the biocellulose material has at least one of
(i) a tensile strength of between about 1 N and about 300 N
(ii) a stiffness of between about 3 N and about 40 N
(iii) a suture pull-out strength of between about 0.3 N and about 15 N.

25. The implantable biocellulose material of claim 23, wherein the material results in a tissue adhesion tenacity of less than about 1.5.
